(19) United States
(12) Patent Application Publication
(10) Pub. No.: US 2023/0158244 A1
(43) Pub. Date: May 25, 2023

(54) INJECTION DEVICES WITH AUTOMATIC PRIMING

(71) Applicant: NDM TECHNOLOGIES LTD, Loughborough (GB)

(72) Inventors: Dewan Fazlul Hoque Chowdhury, Loughborough (GB); Roni Bensimon, London (GB)

(21) Appl. No.: 17/911,863

(22) PCT Filed: Feb. 17, 2021
(86) PCT No.: PCT/GB2021/050381

§ 371 Date: Sep. 15, 2022

(30) Foreign Application Priority Data
Mar. 17, 2020 (GB) 2003866.7

(51) Int. Cl. A61M 5/31

(52) U.S. Cl. CPC A61M 5/3146; A61M 2005/2474

(57) ABSTRACT

An injection device comprises a needle holder that supports a needle, and a vial holder that supports a vial. A spring urges the needle holder to move towards the vial holder but that movement is restrained by a latch until the time of use. Releasing the latch primes the device by allowing the needle holder to move, whereby a proximal end of the needle pierces a septum of the vial. The latch is preferably released by rotating an actuator. The same rotation preferably frees the needle holder and vial to be advanced axially relative to the actuator, whereby a distal end of the needle can be introduced into the skin of a subject. Compressed air from a tank may be used to drive the discharge of the vial. The air tank preferably surrounds the vial and provides a broad proximal surface, against which manual pressure can be applied to the device.

### TECHNICAL FIELD

The invention relates to medical devices for injecting a fluid from a vial, through a needle, into the skin of a subject.

The fluid is typically but not exclusively a biologically active substance such as a pharmaceutical. The subject may be human or animal.

### BACKGROUND OF THE INVENTION

Many injector devices are known, which are used to deliver a fluid into the skin of a subject, while providing additional functionality compared with a manually operated hypodermic syringe. For example, some devices allow a variable dose to be pre-set and others allow the dose to be automatically delivered, for example using compressed air or the stored energy of a spring. In its broadest aspect, the present invention relates to the priming of injector devices prior to their use and is not incompatible with such additional functionality. However, the present invention is normally intended for devices that are single-use, in the sense that an empty vial cannot be replaced with a new one. This does not necessarily exclude using the device, after it has been primed, to deliver multiple doses of the fluid from a single vial.

Biologically active fluids such as pharmaceuticals are frequently packaged in cylindrical vials, which contain an axially sliding plunger. As the plunger moves from the proximal end (closer to the operator) to the distal end (closer to the subject), it displaces fluid from the distal end of the vial. Prior to use, for example during transport and storage, the distal end of the vial is normally sealed by a septum to maintain the sterility of the interior of the vial. Immediately before use, the septum must be ruptured to release the fluid, for example by piercing it with a hollow needle, through which the fluid can be delivered from the vial to the subject. The needle is typically double-ended and is held in a needle hub, which is moved towards the vial until the proximal end of the needle pierces the septum. The distal end of the needle is then available for injection into the skin of the subject. In the present specification, the engagement of the needle hub with the vial so that the needle pierces the septum is referred to as priming the device.

According to the prior art, the needle hub may be manually primed, for example by screwing the needle hub onto a threaded vial holder. It is also known to prime a device semi-automatically with a mechanism that moves the vial towards the needle hub as a result of pressing the device against the skin of the subject.

It is a requirement of injector devices that the distal end of the needle should be shielded from accidental contact before and after use. Many such devices include a retractable shield that surrounds the needle until the device is pressed against the skin of a patient. It is desirable that is should be possible to lock the shield against being retracted accidentally.

Many injection devices have the general form of a hypodermic syringe but on a larger scale to accommodate the additional mechanisms that they contain. The appearance of such “giant syringe” designs can be off-putting for patients who have a fear of injections. They can also cause problems for patients with limited dexterity who need to self-administer injections, who must align the device and support it against the skin, while performing whatever action the particular device requires to cause the delivery of the fluid.

### SUMMARY OF THE INVENTION

The invention provides an injection device as defined in claim 1.

The invention further provides a method of operating such an injection device, as defined in claim 15.

Features of the invention that are preferred but not essential are defined in the dependent claims.

### THE DRAWINGS

FIG. 1 is a perspective view of an injector device according to the invention.

FIG. 2 is the same view as FIG. 1 with the air tank removed.

FIG. 3 is a longitudinal cross section through the injector device of FIG. 1 after it has been primed.

FIG. 4 is a perspective view from above, showing the relationship between the rotary actuator and the needle holder.

FIG. 5 is a longitudinal cross section, showing the relationship between the rotary actuator and the needle holder in the latched condition of the device.

FIG. 6 is a perspective view from above, showing the relationship between the rotary actuator and the vial holder.

FIG. 7 is a longitudinal section through the vial holder on plane A-A of FIG. 6.

FIG. 8 is a longitudinal cross section similar to FIG. 3, showing the needle of the injector device in an advanced position.

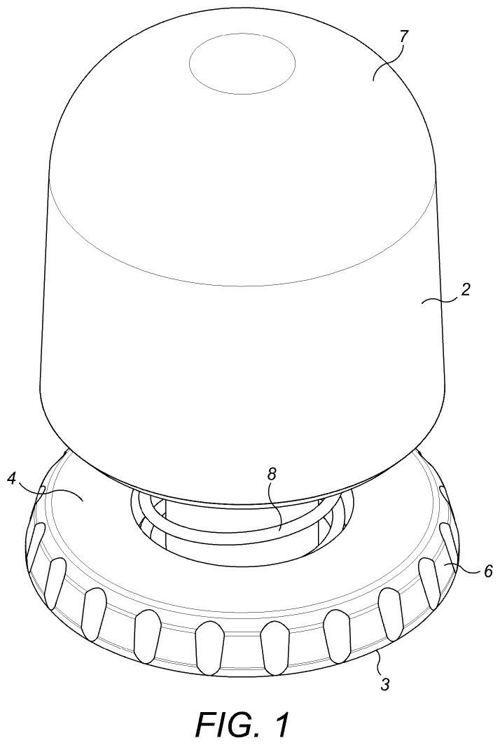

FIG. 1 shows an injector device in accordance with the present invention. The upper part of the device comprises a domed compressed air tank 2. The lower part of the device comprises a rotary actuator 4 having a wide base 3 and a central upstanding boss 5. As will be described below, the device may be primed by gripping the actuator 4 on its knurled rim 6 and rotating it through a predetermined angle relative to the air tank 2. The base 3 of the actuator 4 may then be rested against the skin of a subject and an injection can be performed by applying pressure to the broad upper surface 7 of the air tank 2 to move the tank towards the base 3 against the force of a compression spring 8. A needle 9 on the axis of the device (not seen in FIG. 1) is thereby lowered through an aperture in the base 3 to penetrate the surface of the skin.

The device in this embodiment of the invention has the overall appearance and function of a large push-button. Its rounded shape, being very different in appearance from a conventional hypodermic syringe or injector pen, is less threatening to many subjects who have a fear of needles. It is also very easy and reliable to use in a “twist and press” action. After the device has been primed by twisting the base 3 relative to the air tank 2, the injection can then be performed with one hand. Operating the push-button requires little dexterity, and the wide base 3 keeps the device stable, ensuring that its axis remains perpendicular to the skin.

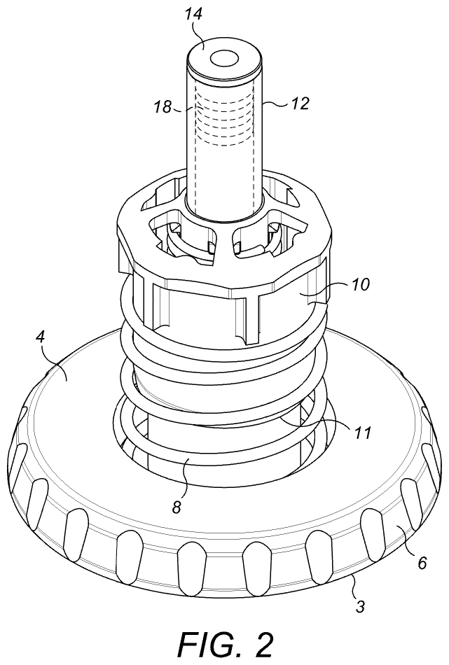

FIG. 2 is a similar view to FIG. 1 but with the air tank 2 removed to reveal more details of the device. It can be seen that the spring 8 acts between the actuator 4 and a vial holder 10, which can move axially relative to the actuator 4. The vial holder 10 can move through a predetermined distance before a distal edge 11 of the vial holder butts against the base 3 of the actuator 4. The vial holder 10 supports a vial 12 of the fluid that is to be delivered by the device. The upper (proximal) end of the vial 12 is exposed by the vial holder 10 and is closed by a vial cap 14. The lower (distal) end of the vial 12 (not seen in FIG. 2) is sealed by a septum 16 to maintain the contents of the vial sterile during transportation and storage. When pressure is applied to a plunger 18 within the vial 12, it can slide axially in the distal direction to displace fluid from the distal end of the vial 12.

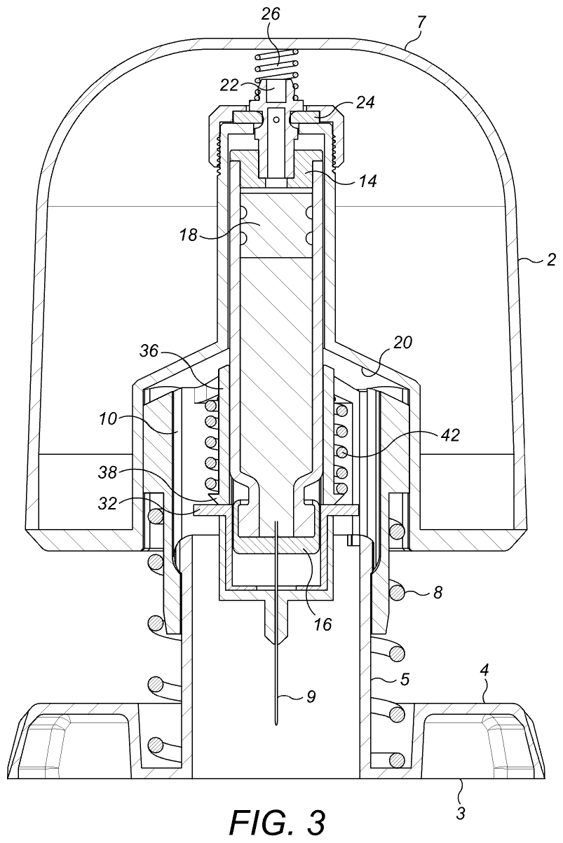

FIG. 3 shows a cross section on the axis of the injector device of FIGS. 1 and 2. It shows how the vial holder 10 slides axially on an outer surface of the boss 5. It also shows how an inner wall 20 of the air tank 2 is shaped to surround the vial 12 and to be capable of sliding axially on the periphery of the vial holder 10. A valve 22 pierces an upper end of the inner wall 20 and provides a conduit for the selective release of compressed air from the tank 2 when a valve seal 24 is ruptured. The valve 22 is connected to an aperture in the vial cap 14 so that it can deliver compressed air into a space between the vial cap 14 and the plunger 18, which will drive the plunger in the distal direction. When the air tank 2 slides on the vial holder 10 and moves axially relative to the vial 12, the valve remains fixed in relation to the vial cap 14. This relative movement between the air tank 2 and the valve 22 is capable of rupturing the valve seal 24 and releasing compressed air from the tank 2 into the vial 12. A compression spring 26 acting between the air tank 2 and the valve 22 offers resistance to the relative movement between them. This valve spring 26 has a higher spring constant than the actuator spring 8, whereby, when a given force is applied to the proximal surface 7 of the air tank 2, the vial holder 10 will move fully through the predetermined axial distance before there is sufficient relative movement between the air tank 2 and the valve 22 to rupture the valve seal 24.

FIG. 3 also shows a needle holder 32. The needle holder 32 supports the needle 9, which is aligned with the axis of the device. A distal end of the needle 9 can be moved along the axis to penetrate the skin of a subject, as previously described. A proximal end of the needle 9 can be moved in the proximal direction to pierce the septum 16 of the vial 12, as seen in FIG. 3. The hollow needle 9 thereby provides a conduit for fluid to flow from the vial 12 into the skin of the subject. In this embodiment, the needle 9 is supported directly by the needle holder 32. It would also be possible for the needle 9 to be supported in a needle hub (not shown), which is in turn mounted on the needle holder. For example, the needle hub could be generally cup-shaped, being concave in the proximal direction, and push-fitted onto a complementary structure at the distal end of the needle holder 32.

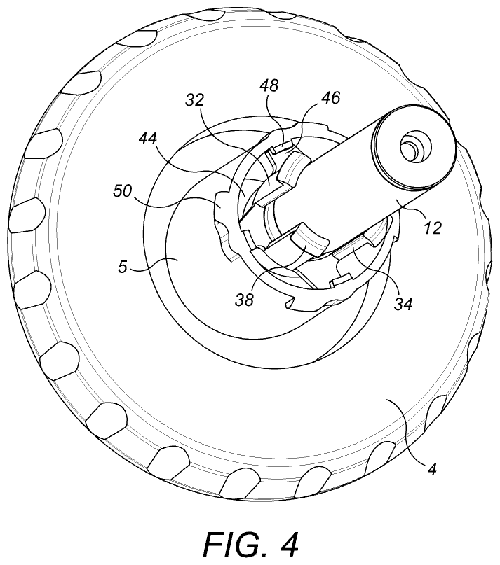

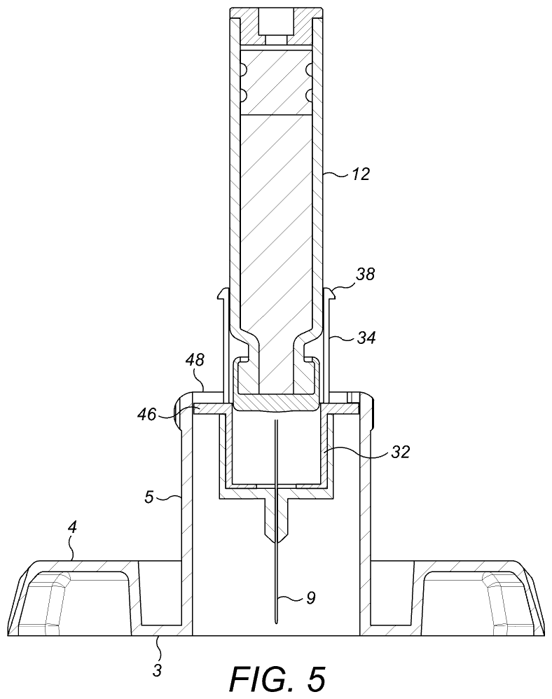

The needle holder 32 is in turn supported by the vial holder 10. The needle holder 32 comprises four hub arms 34 (seen in FIGS. 4 and 5) that are circumferentially spaced and extend axially from the needle holder 32 in the proximal direction. The needle holder 32 comprises four hub arms 34 (seen in FIGS. 4 and 5) that are circumferentially spaced and extend in the proximal direction from the needle holder 32. The vial holder 10 comprises four complementary vial arms 36 that are circumferentially spaced and extend in the distal direction from the proximal end of the vial holder 10. (The vial arms 36 can be seen through openings in the proximal end of the vial holder 10 in FIG. 2.) The hub arms 34 and the vial arms 36 interdigitate to form between them a complete collar around the vial 12 in the region where they overlap. They thus prevent relative rotational movement between the needle holder 32 and the vial holder 10 but they permit a degree of relative axial movement as the arms 34,36 slide past one another. It will be understood that the number of hub arms 34 and the number of vial arms 36 could be different from four.

The tips of the hub arms 34 project radially outwards to form a hub spring seat 38 and the tips of the vial arms 36 project radially outwards to form a vial spring seat 40. A compression spring 42 acts between the two spring seats 38,40 and urges them axially apart. By urging the tips of the interdigitating hub arms 34 and vial arms 36 further apart, the tendency of the spring 42 is to increase the length of overlap between the arms 34,36. If the needle holder 32 is free to move, the spring 42 therefore draws it in the proximal direction until the needle holder 32 abuts the vial holder 10 and the proximal end of the needle 9 has pierced the septum 16 of the vial 12. The spring 42 thereafter holds the needle holder 32 and the vial holder 10 in abutment and they are effectively become locked together both axially and rotationally.

However, in accordance with the invention, it is required that the needle 9 should be prevented from piercing the septum 16 until the device is primed immediately before use. Accordingly, a latch mechanism is provided to restrain the axial movement of the needle holder 32 until the latch is released.

In the illustrated embodiment of the invention, the latch is provided by the rotary actuator 4. FIG. 4 shows how the needle holder 32 sits in a central aperture 44 of the boss 5 of the actuator 4. Four circumferentially spaced hub holder lugs 46 project radially outwards from the needle holder 32. Four circumferentially spaced actuator lugs 48 project radially inwards from the actuator 4. When the actuator 4 is in a first angular position, as shown in FIG. 4, the actuator lugs 48 interfere with the hub holder lugs 46 to obstruct the passage of the needle holder 32 through the aperture 44. Therefore, despite the action of the spring 42 urging the needle holder 32 in the proximal direction, it is restrained at the axial position shown in FIG. 5. When the needle holder 32 is latched in this position, the proximal end of the needle 9 does not pierce the septum 16 and the distal end of the needle 9 does not project beyond the base 3 of the actuator 4 so the device can remain safe and sterile during transport and storage. When the actuator 4 is manually rotated through a sufficient angle for the respective lugs 46,48 to pass each other, the needle holder 32 becomes free to move automatically under the influence of the spring 42. This causes the needle 9 to pierce the septum 16 and prime the device for use.

It will be understood that the number of holder lugs 46 and the number of actuator lugs 48 does not have to be four; and it is not strictly essential that the numbers should be equal, provided that there are some rotary positions where the respective lugs 46,48 interfere to prevent the passage of the needle holder 32 through the aperture 42. More generally, the aperture 42 can have almost any non-circular cross-section and it will be possible to design a profile of the needle holder 32 that will fit through it in certain angular positions but not in other angular positions. (Neither the aperture nor the profile can have circular symmetry about the axis.) It is preferred that the actuator 4 should not need to be rotated through a large angle to release the latch. For example, the angle is preferably less than 45° and more preferably 15° to 20°.

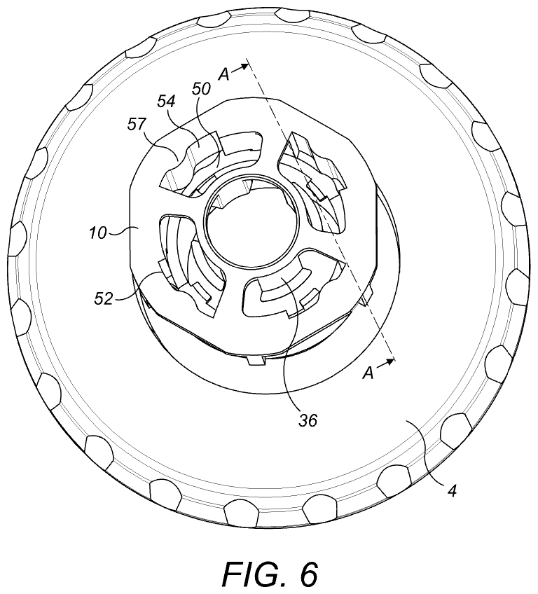

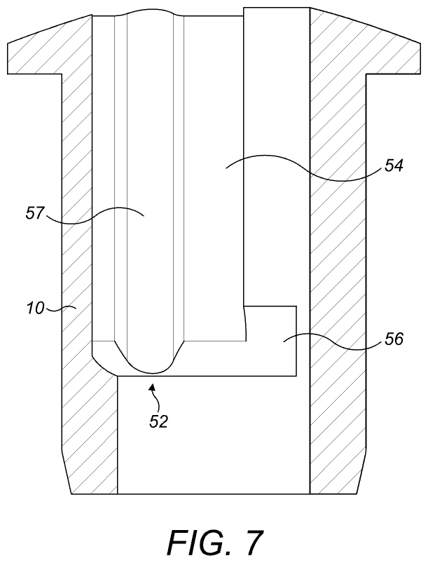

Rotation of the actuator 4 relative to the needle holder 32 is permitted by the engagement between the actuator 4 and the vial holder 10. (It will be recalled that the vial holder 10 and the needle holder 32 are rotationally locked together by their respective interdigitating arms 34,36.) As seen in FIG. 4, four circumferentially spaced protrusions 50 project radially outwards from the periphery of the boss 5 of the actuator 4. Each protrusion 50 is located in a guide track 52 on a cylindrical inner surface of the vial holder 10. FIGS. 6 and 7 show that each guide track 52 follows a substantially L-shaped course, comprising a linear part 54 that is parallel to the axis and a circumferential part 56 that intersects with the distal end of the linear part 54. When the protrusions 50 of the actuator 4 follow the circumferential parts 56 of the guide tracks, they permit relative rotation but not relative axial movement between the actuator 4 and the vial holder 10. When the protrusions 50 of the actuator 4 follow the linear parts 54 of the guide tracks, they permit relative axial movement but not relative rotation between the actuator 4 and the vial holder 10.

When the protrusions 50 of the actuator 4 are located at the blind ends of the circumferential parts 56 of the guide tracks, this corresponds to the rotary actuator 4 being in its first position, in which the actuator lugs 48 obstruct the holder lugs 46 to restrain the needle holder 32 against axial movement. At the same time, the shape of the guide track 52 prevents relative axial movement between the actuator 4 and the vial holder 10. Therefore, in the first position, not only is the needle holder 32 latched to prevent priming of the device but the operation of the “push-button” is also latched to prevent accidental deployment of the injector needle 9.

FIG. 6 shows the profile of the linear parts 54 of the guide tracks 52, which is complementary to the profile of the protrusions 50. (In FIG. 6, the protrusions 50 are partly rotated out of view in the circumferential parts 56 of the guide tracks 52.) It can be seen that the profile includes a “bump” 57, which offers slight resistance when the actuator 4 is rotated to or from the first position and provides haptic and/or audible feedback in the form of a click when the device is latched or unlatched.

As the actuator 4 is rotated from its first position to its second position, the needle holder 32 becomes unlatched and moves under the influence of the spring 42 to prime the device for use. The same rotation causes the protrusions 50 to follow the circumferential parts 56 of the guide tracks 52 until they reach the intersection with the linear parts 54. In this second position, the vial holder 10 becomes free to move axially relative to the actuator 4 and the device can be operated to inject the fluid into the skin of a subject. If desired, an intermediate angular position of the actuator 4 could be defined between the first and second positions, at which the device has been primed but the push-button has not yet been unlatched. A second bump could be provided in the profile of the circular parts 56 of the guide tracks to hold the actuator 4 in that intermediate position and provide further haptic and/or audible feedback to the user.

Note that it would be possible to invert the illustrated arrangement so that protrusions from the vial holder 10 follow guide tracks in the actuator 4.

In the light of the foregoing explanation, the operation of the device should be apparent but will now be briefly described.

During manufacture, the device is assembled around a sealed and sterile vial 12. The actuator 4 is rotated to the first position to latch the device against premature priming or operation during transport and storage. The needle holder 32 is restrained in the position shown in FIG. 5.

When it is desired to deliver the dose of fluid from the vial to a subject, the device is first primed by rotating the actuator 4 relative to the via holder 10 from its first position to its second position. This unlatches the needle holder 32, which moves under the influence of the spring 42 to cause the proximal end of the needle 9 to pierce the septum 16 of the vial 12. This is the position shown in FIG. 3.

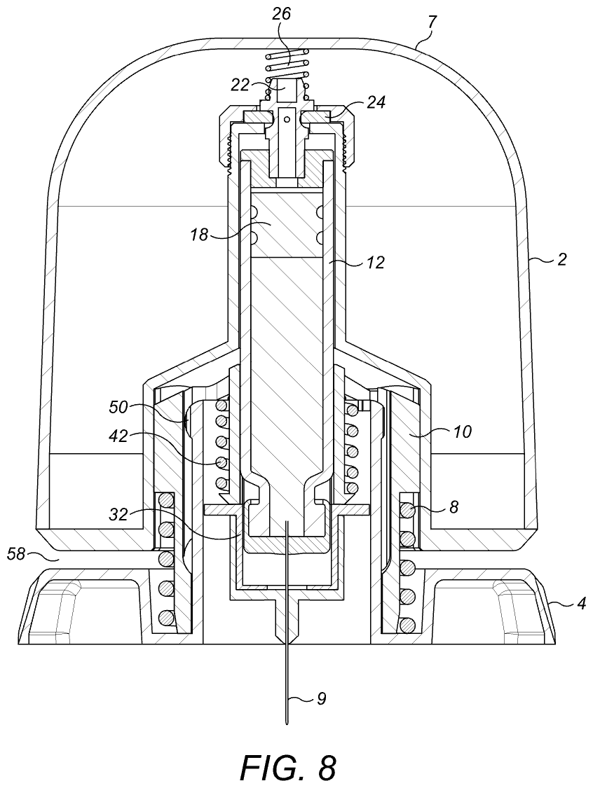

The base 3 of the actuator is then placed against the skin of a subject and sufficient pressure is applied to the proximal surface 7 of the compressed air tank 2 to overcome the resistance of the actuator spring 8. The air tank 2, vial holder 10, vial 12 and needle holder 32 move axially as a unit, sliding on the boss 5 of the actuator 4 until the bottom edge 11 of the vial holder 10 abuts the base 3 of the actuator 4 and the distal end of the needle 9 has penetrated the skin of the subject. In this position the vial holder 10 can move no further but, as shown in FIG. 8, there remains a small gap 58 between the bottom of the compressed air tank 2 and the base 3 of the actuator 4. Further pressure applied to the proximal surface 7 of the air tank 2 can overcome the resistance of the valve spring 26 and cause further axial movement of the air tank 2 relative to the vial holder 10 and the valve 22, to close the gap 58. The relative movement between the air tank 2 and the valve 22 causes the valve seal 24 to rupture and releases compressed air from the tank 2 into the vial 12, thereby driving the plunger 18 in the distal direction and displacing fluid from the vial 12 through the hollow needle 9 and into the skin of the subject.

The invention has been described with reference to a single embodiment but there is no intention to limit the scope of the invention to that embodiment of it. The reader will understand different ways of putting the invention into practice. It should also be understood that different aspects of the illustrated embodiment can be used independently of each other. In particular, the means for priming the device by unlatching it, to allow an internal spring 42 to drive the needle 9 to pierce the septum 16, could be used with a different means for operating the device to deliver a dose of fluid to the subject.

1. An injection device comprising:
a needle, which is hollow and which comprises a proximal end and a distal end;
a needle holder, which supports the needle;
a vial, comprising a distal end that is sealed by a septum;
a vial holder, which supports the vial;
a first spring, which acts between the vial holder and the needle holder to urge the needle holder to move in the proximal direction relative to the vial holder, whereby the proximal end of the needle pierces the septum of the vial;
an actuator; and
a latch, which releasably restrains the needle holder against movement in the proximal direction;
wherein the first spring is compressed between a vial spring seat on the vial holder and a needle spring seat on the needle holder, the needle spring seat being in the proximal direction from the vial spring seat,
wherein the vial spring seat and the needle spring seat are respectively provided on interdigitating parts of the vial holder and the needle holder.

2. (canceled)

3. (canceled)

4. An injection device according to claim 1, wherein the latch operates between the actuator and the needle holder, the actuator being rotatable about the axis relative to the needle holder between a first position in which the actuator restrains the needle holder against movement in the proximal direction and a second position in which the actuator does not restrain the needle holder against movement in the proximal direction.

5. An injection device according to claim 1, wherein:
the actuator defines a non-circular aperture; and
the needle holder has a non-circular profile;
wherein the non-circular profile of the needle holder cannot pass through the aperture when the actuator is in the first position but can pass through the aperture when the actuator is in the second position.

6. An injection device according to claim 5, wherein the actuator comprises a plurality of circumferentially spaced actuator lugs that project radially inwards into the aperture to make it non-circular; and the needle holder comprises a plurality of circumferentially spaced holder lugs that project radially outwards to form the non-circular profile.

7. An injection device according to claim 4, wherein the actuator is rotatably mounted in the vial holder.

8. An injection device according to claim 7, wherein the actuator engages the vial holder such that:
from the first position the actuator is able to rotate towards the second position but not move axially relative to the vial holder; and
from the second position the actuator is able to move axially in the proximal direction through a predetermined distance relative to the vial holder.

9. An injection device according to claim 8, wherein one of the actuator and the vial holder comprises a protrusion that is constrained to move within a guide track on the other of the actuator and the vial holder.

10. An injection device according to claim 8, further comprising a second spring that acts between the actuator and the vial holder to resist the axial movement of the actuator in the proximal direction relative to the vial holder.

11. An injection device according to claim 10, further comprising:
a vial cap that closes the proximal end of the vial;
a plunger mounted for axial movement within the vial;
a compressed air tank; and
a valve, which selectively provides fluid communication from the air tank to a space between the vial cap and the plunger;
wherein the valve is configured to open when a predetermined force is applied to a proximal surface of the device, that force being greater than the force required to move the actuator through the predetermined distance against the resistance of the second spring.

12. An injection device according to claim 11, wherein:
an external surface of the air tank forms the proximal surface of the device;
the air tank is mounted to be capable of axial movement relative to the valve; and
the air tank engages the valve such that, when the predetermined force is applied to the proximal surface of the device, the air tank moves axially and causes the valve to open.

13. An injection device according to claim 12, further comprising a third spring that acts between the air tank and the valve.

14. An injection device according to claim 12, wherein the air tank surrounds the vial and the axial movement of the air tank is guided by the vial holder.

15. A method for operating an injection device according to claim 4, comprising:
priming the device by rotating the actuator from the first position to the second position, whereby the latch releases the needle holder and the first spring moves the needle holder in the proximal direction until the proximal end of the needle pierces the septum of the vial.

16. A method for operating an injection device according to claim 8, comprising:
priming the device by rotating the actuator from the first position to the second position, whereby the latch releases the needle holder and the first spring moves the needle holder in the proximal direction until the proximal end of the needle pierces the septum of the vial; and
advancing the needle by applying pressure between opposite axial ends of the device to cause the actuator to move through the predetermined distance relative to the vial holder, whereby the distal end of the needle projects beyond a distal surface of the actuator.

17. (canceled)

18. A method according to claim 16, wherein the step of applying pressure between opposite axial ends of the device includes pressing the distal surface of the actuator against the skin of a subject.

19. A method for operating an injection device according to claim 11, comprising:
priming the device by rotating the actuator from the first position to the second position, whereby the latch releases the needle holder and the first spring moves the needle holder in the proximal direction until the proximal end of the needle pierces the septum of the vial;
advancing the needle by applying pressure between opposite axial ends of the device to cause the actuator to move through the predetermined distance relative to the vial holder, whereby the distal end of the needle projects beyond a distal surface of the actuator; and
discharging the vial by applying the predetermined force to the valve, whereby compressed air enters the space between the vial cap and the plunger and drives the plunger in the distal direction.
